(19) United States
(12) Patent Application Publication
(10) Pub. No.: US 2026/0018260 A1
(43) Pub. Date: Jan. 15, 2026

(54) METHOD AND DEVICE FOR EVALUATING DYSARTHRIA

(71) Applicant: HAIl CORP., Seoul (KR)

(72) Inventors: Taejin SONG, Seoul (KR); Yuyoung KIM, Yongin-si (KR); Sangkwon LIM, Guri-si (KR); Saebyeol KIM, Seoul (KR); Minjung KIM, Seongnam-si (KR); Hyungmin PARK, Seoul (KR); Unsang PARK, Seoul (KR); Myoungwan KOO, Seoul (KR); Jihwan KIM, Seoul (KR)

(21) Appl. No.: 18/997,874

(22) PCT Filed: Oct. 24, 2023
(86) PCT No.: PCT/KR2023/016588

§ 371 Date: Jan. 23, 2025

(30) Foreign Application Priority Data
Dec. 8, 2022 (KR) 10-2022-0170565

(51) Int. Cl. G16H 10/20; A61B 5/00

(52) U.S. Cl. CPC G16H 10/20; A61B 5/4803

(57) ABSTRACT

The present disclosure relates to a method which a server receives voice data of a user from a user terminal and evaluates dysarthria of the user, the server including a processor and a database and communicates with the terminal, and the terminal including a processor, a memory, a display, and a microphone and communicating with the server. The method comprises steps in which the server: causing the terminal to describe a dysarthria evaluation process to the user; causing the user terminal to check whether the surrounding environment of the user is suitable to evaluate the voice of the user; causing the user terminal to induce the user to perform at least one from among sustained phonation, articulatory diadochokinesis, word reading, and sentence reading for sustained phonation evaluation, and record the voice of the user; and evaluating degrees of dysarthria of the user on the basis of the recorded voice data.

### TECHNICAL FIELD

The present disclosure relates to a method for evaluating dysarthria, and more particularly to a method for conveniently evaluating dysarthria of a user with high accuracy.

### BACKGROUND ART

Dysarthria is a speech disorder in which muscle control of the speech production mechanism is impaired due to damage to the central or peripheral nervous system, resulting in weakened or slowed muscle movement ability and poor coordination.

There is a matter that, in order to evaluate the presence or absence of speech disorders such as dysarthria and the severity of the disorder, a user needs to directly meet and speak to an evaluator so that his or her speech is evaluated.

Here, in recent years, due to the COVID pandemic, there is a need for a method by which a large number of subjects to be evaluated can be evaluated for the presence or absence of speech disorders and the severity of the disorder without having to meet evaluators. However, useful methods are not presented.

Also, in order to improve dysarthria caused due to various reasons such as brain damage, speech diagnosis (evaluation) and treatment are currently performed by speech-language pathologists (SLPs) on the basis of a speech therapy. A speech therapy performed by SLP is performed 2-3 times a week. In addition, because it is performed by SLP, there are problems in that the evaluation and treatment costs are high and the evaluation criteria may vary in accordance with the therapist.

### CITATION LIST

### Patent Literature

Korean Patent Publication No. 10-2022-0128976

### DETAILED DESCRIPTION OF INVENTION

### Technical Problem

An object of the present disclosure is to provide a new solution in which many people solve a matter of needing to evaluate their dysarthria by providing a dysarthria evaluation method which allows dysarthria to be evaluated conveniently with high accuracy.

### Solution to Problem

According to an aspect of the present disclosure, provided is a method for evaluating dysarthria by which a server receives voice data of a user from a user terminal and evaluates dysarthria of the user, wherein the server includes a processor and a database and communicates with the terminal, and the terminal includes a processor, a memory, a display, and a microphone and communicates with the server, the method comprises steps in which the server: causing the terminal to describe a dysarthria evaluation process to the user; causing the user terminal to check whether the surrounding environment of the user is suitable to evaluate the voice of the user; causing the user terminal to induce to perform at least one from among sustained phonation, articulatory diadochokinesis, word reading, and sentence reading for sustained phonation evaluation, and record the voice of the user; and evaluating a degree of dysarthria of the user on the basis of the recorded voice data, and the sustained phonation evaluation includes evaluating a degree of breathing insufficiency of the user, a level of breathing intensity, a degree of breathing intensity reduction, and a variation in breathing intensity to evaluate the breathing of the user.

In an example embodiment, the step of checking whether the surrounding environment of the user is suitable to evaluate the voice of the user may include causing the server to cause the terminal to record noise around the user and generate sound data; causing the server to receive the generated sound data from the terminal; causing the server to receive the sound data from the terminal; causing the server to analyze the received sound data to evaluate a level of noise around the user; and causing the server to transmit, to the user terminal, information regarding whether the surrounding environment is suitable to evaluate the voice of the user on the basis of the result of evaluating the level of noise.

In an example embodiment, the sustained phonation evaluation may include an evaluation of at least one of the respiration of the user, phonation, resonance, prosody, and articulation.

In an example embodiment, the sustained phonation evaluation may include evaluating a degree of breathing insufficiency of the user, a level of breathing intensity, a degree of breathing intensity reduction, and a variation in breathing intensity to evaluate the breathing of the user.

In an example embodiment, the sustained phonation evaluation may include evaluating a pitch level, a vocalization intensity level, sound quality, and pitch variations to evaluate the vocalization of the user.

In an example embodiment, the sustained phonation evaluation may include evaluating a degree of nasality and weak plosives to evaluate the resonance of the user.

In an example embodiment, the sustained phonation evaluation may include evaluating a speech rate, regularity, and inappropriate pauses to evaluate the rhyme of the user.

In an example embodiment, the sustained phonation evaluation may include evaluating consonant accuracy and vowel accuracy to evaluate the articulation of the user.

In an example embodiment, before the step of checking whether the surrounding environment of the user is suitable to evaluate the voice of the user, the method may further include causing the server to cause the user terminal to conduct a questionnaire to evaluate a psychological state of the user.

In an example embodiment, the method further include: after the step of evaluating a degree of dysarthria of the user on the basis of the recorded voice data, causing the server to transmit an evaluation result report including evaluation result information obtained by evaluating a degree of dysarthria of the user to the user terminal; and causing the server to cause the user terminal to display the evaluation result report on the display and report the evaluation result report to the user.

In an example embodiment, the evaluation result report may include the contents of classifying the ability of at least one of the respiration of the user, phonation, resonance, prosody, and articulation as high, average, or low.

In an example embodiment, the evaluation result report may include a graph showing the ability of at least one of the respiration of the user, phonation, resonance, prosody, and articulation.

In an example embodiment, the evaluation result report may include the contents indicating a port of the sentence read by the user in which inappropriate pauses appear to evaluate the inappropriate pauses in the prosody of the user.

In an aspect of the present disclosure, a dysarthria evaluation device performing the method for evaluating dysarthria includes a processor and a memory.

### Advantageous Effects of Invention

A method for evaluating dysarthria according to an example embodiment of the present disclosure includes evaluating a degree of dysarthria of a user on the basis of recorded voice data, thereby enabling the degree of dysarthria of the user to be conveniently evaluated with high accuracy.

### BRIEF DESCRIPTION OF DRAWINGS

FIG. 1 is a schematic diagram showing a server and a terminal in which a method for evaluating dysarthria according to an example embodiment of the present disclosure is used.

FIG. 2 is a flowchart for describing steps in the method for evaluating dysarthria according to an example embodiment of the present disclosure.

FIGS. 3 to 18 are images showing the details of a display of a terminal in which a method for evaluating dysarthria according to an example embodiment of the present disclosure.

FIG. 19 is a table showing evaluation items, diagnostic tasks, and detailed evaluation items used for performing a method for evaluating dysarthria according to an example embodiment of the present disclosure.

FIG. 20 is a table showing evaluation criteria for a maximum phonation time (MPT) used for performing a method for evaluating dysarthria according to an example embodiment of the present disclosure.

FIG. 21 is a table showing evaluation criteria for a pitch level of vocalization used for performing a method for evaluating dysarthria according to an example embodiment of the present disclosure.

FIG. 22 is a table showing evaluation criteria for pitch variations used for performing a method for evaluating dysarthria according to an example embodiment of the present disclosure.

FIG. 23 is an image showing the details of a display of a terminal in which a method for evaluating dysarthria according to an example embodiment of the present disclosure is used.

FIG. 24 is an image showing the details of a display of a terminal in which a method for evaluating dysarthria according to an example embodiment of the present disclosure is used.

FIGS. 25 to 40 are images showing the details of an evaluation result report provided in a method for evaluating dysarthria according to an example embodiment of the present disclosure.

### DESCRIPTION OF EMBODIMENTS

The advantages and the features of the present disclosure and the methods for achieving them will become apparent with reference to the embodiments which will be described below in detail together with the attached drawings. Here, the present disclosure is not limited to the embodiments which will be disclosed below and may be implemented in various different forms and these embodiments are provided only to ensure complete disclosure of the present disclosure and to fully inform a person skilled in the art to which the present disclosure belongs of the scope of the invention and the present disclosure is defined only by the scope of the claims.

The terminology used herein is for the purpose of describing particular embodiments only and is not intended to limit the present disclosure. For example, a constituent element expressed in the singular needs to be understood as a concept including plural constituents unless the context clearly indicates that only the singular is meant. Furthermore, in the specification of the present disclosure, terms such as ‘comprise,’ ‘include,’ and ‘have’ are intended only to specify the presence of features, numbers, steps, operations, constituent elements, parts, or combinations thereof described in the specification. In addition, the use of these terms does not exclude the presence or addition of one or more other features, numbers, steps, operations, constituent elements, parts or combinations thereof.

In addition, unless otherwise defined, all terms used herein, including technical or scientific terms, have the same meaning as commonly understood by a person of ordinary skill in the art to which the present disclosure belongs.

Terms such as those defined in commonly used dictionaries need to be interpreted as having a meaning consistent with their meaning in the context of the relevant technology. In addition, it shall not be construed in an idealistic or overly formal sense unless expressly defined in the specification of the present disclosure.

Embodiments of the present disclosure will be described in more detail below with reference to the attached drawings. Here, in the following description, specific descriptions of widely known functions or configurations are omitted if they may unnecessarily obscure the gist of the present disclosure.

The literature in the related art described in the present disclosure are incorporated herein by reference in their entirety and it will be understood that the teachings of the literature in the related art could have been applied by a person of ordinary skill in the art to the matters described in the present disclosure.

A device and a method 100 for improving dysarthria according to an example embodiment of the present disclosure will be described below with reference to the drawings.

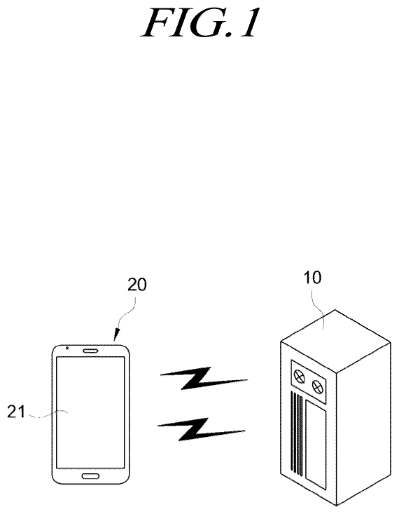

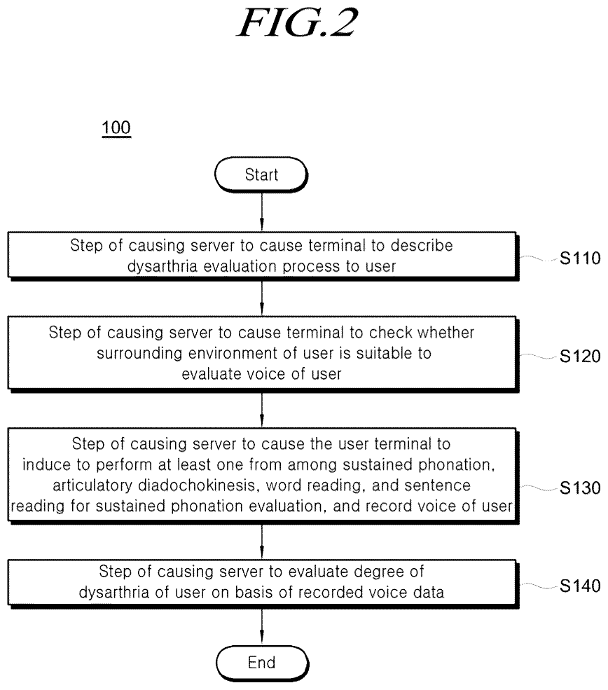

FIG. 1 is a schematic diagram showing a server 10 and a terminal 20 in which the method 100 for evaluating dysarthria according to an example embodiment of the present disclosure is used. Moreover, FIG. 2 is a flowchart for describing steps S110 to S140 of the method 100 for evaluating dysarthria according to the example embodiment of the present disclosure.

Referring to FIGS. 1 and 2, the method 100 for evaluating dysarthria of the present disclosure is a method 100 for evaluating dysarthria in which the server 10 receives voice data of a user from a user terminal 20.

The server 10 may include a processor and a database and communicate with the terminal 20. The server 10 is configured to analyze the received voice data of the user and generate a dysarthria evaluation result to be provided to the user on the basis of the analysis.

The server 10 can analyze the voice data of the user to obtain, for example, at least one of the voice volume (decibels), pitch, pronunciation accuracy, sound length, pitch change, breath holding, beat, and reading speed of the user. The method 100 for analyzing the voice data of the user will be described in detail later.

The server 10 includes a processor, a communication module (not shown), and a memory (not shown).

For example, a communication method for the communication module may utilize a network built according to, but is not limited to, Global System for Mobile (GSM) communication, Code Division Multi Access (CDMA), High Speed Downlink Packet Access (HSDPA), High Speed Uplink Packet Access (HSUPA), Long Term Evolution (LTE), Long Term Evolution-Advanced (LTE-A), or the like), Wireless LAN (WLAN), Wireless-Fidelity (Wi-Fi), Wireless Fidelity (Wi-Fi) Direct, Digital Living Network Alliance (DLNA), Wireless Broadband (WiBro), World Interoperability for Microwave Access (WiMAX), 5-Generation (G), or the like, and may include all transmission method standards to be developed in the future. It can include anything which can transmit and receive data via wired or wireless means. Scripts stored in memory and visual information corresponding to the scripts may be updated through the communication module.

The memory is configured to store instructions to be performed by a processor (not shown). The memory may be configured to store voice data and an evaluation result.

In an example embodiment, a memory may include computer-readable storage media such as data storage devices which are accessible by the computing device and provide persistent storage of data and executable instructions (for example, software applications, programs, functions, or the like). Examples of the memory include volatile memories and non-volatile memories, fixed and removable media devices, and any suitable memory device or electronic data storage having maintains data for access by the computing device held therein. The memories may include various embodiments of random access memories (RAMs), read-only memories (ROMs), flash memories, and other types of storage media in various memory device configurations. The memory may be configured to store executable software instructions (for example, computer-executable instructions) which are executable together with the processor or the same software application which may be implemented as a module.

The criteria for the dysarthria evaluation items provided by server 10 may be stored in a memory.

The terminal 20 may be configured to communicate with the server 10. The terminal 20 includes a processor (not shown), a memory (not shown), a display 21, a speaker (not shown), and a microphone (not shown).

The processor may be configured to perform tasks according to instructions stored in the memory.

The microphone may be configured to detect the speech of the user to record the voice of the user.

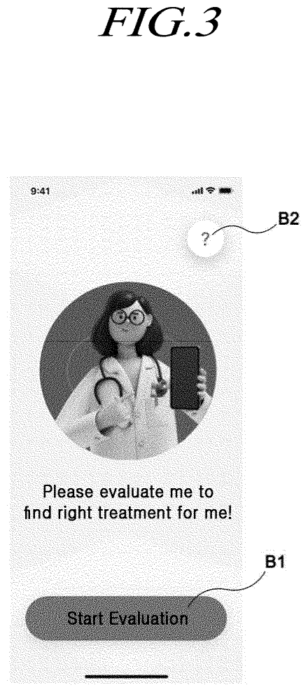

FIGS. 3 to 18 are images showing the details of the display of the terminal 20 in which the method 100 for evaluating dysarthria according to the example embodiment of the present disclosure is used.

The method 100 for evaluating dysarthria of the present disclosure may cause the server 10 to cause the terminal 20 to display a button B1 configured to starting dysarthria evaluation (evaluate) and a button B2 configured to explain the cause and the process of dysarthria to the user. For example, the method 100 for evaluating dysarthria of the present disclosure may cause the server 10 to cause the terminal 20 to display the button B1 having a label of “Start voice evaluation” and the button B2 configured to describe the cause and the process of dysarthria having a label of “?,” as in FIG. 3.

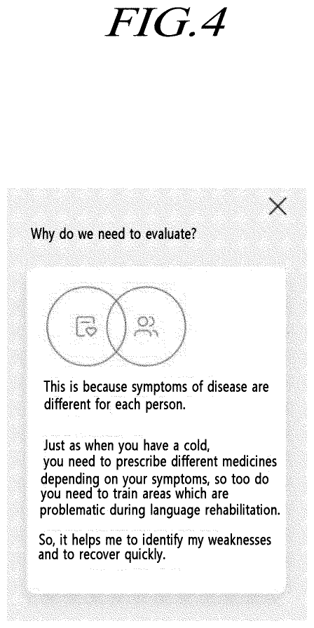

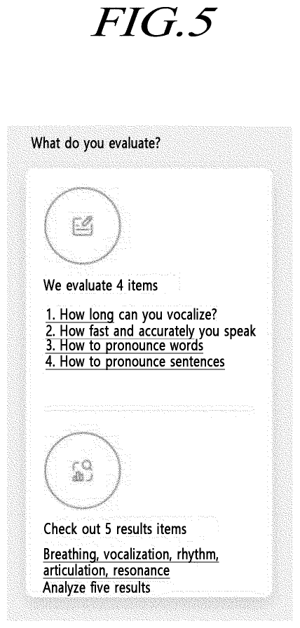

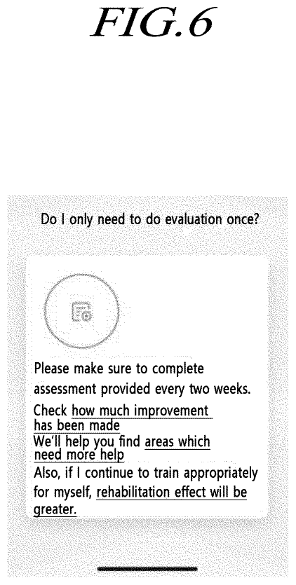

The method 100 for evaluating dysarthria of the present disclosure includes a step S110 of causing the server 10 to cause the terminal 20 to describe a dysarthria evaluation process to the user in response to the pressing of the user of the button B2 having a “?” label. At this time, the server 10 may cause the terminal 20 to display text describing the dysarthria evaluation process. For example, as in FIG. 4, the method 100 for evaluating dysarthria of the present disclosure may cause the server 10 to cause the display of the terminal 20 to display text describing the need to evaluate dysarthria. For example, as in FIG. 4, the method 100 for evaluating dysarthria of the present disclosure may cause the server 10 to cause the display of the terminal 20 to display text describing the need to evaluate dysarthria (for example, “Why should we evaluate it?”). For example, as in FIG. 5, the method 100 for evaluating dysarthria of the present disclosure may cause the server 10 to cause the display of the terminal 20 to display text describing dysarthria evaluation items (for example. “What are you evaluating?”). For example, as in FIG. 6, the method 100 for evaluating dysarthria of the present disclosure may cause the server 10 to cause the display of the terminal 20 to display text describing the dysarthria evaluation cycle (e.g., “Do I only need to evaluate once?”).

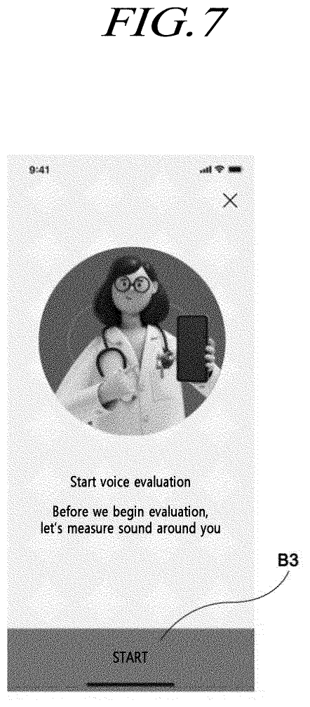

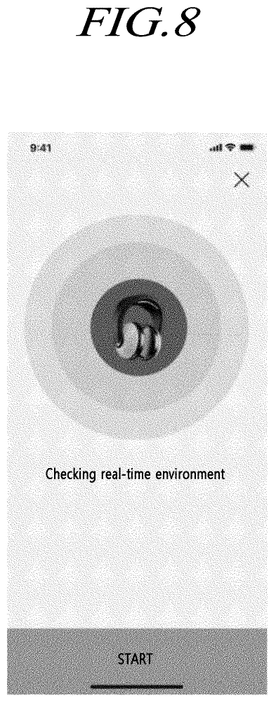

The method 100 for evaluating dysarthria of the present disclosure may cause the server 10 to perform a step S120 of causing the user terminal 20 to check whether the surrounding environment of the user is suitable to evaluate the voice of the user in response to the selecting of the user of the button for starting voice evaluation as shown in FIG. 7. For example, as in FIG. 7, when the user presses a start button B3 configured to start voice evaluation displayed on the display of the terminal 20, as in FIG. 8, the server 10 can perform a step S120 of causing the user terminal 20 to check whether the surrounding environment of the user is suitable to evaluate the voice of the user.

Specifically, the step S120 of checking whether the surrounding environment of the user is suitable to evaluate the voice of the user includes a step of causing the server 10 to cause the terminal 20 to record the surrounding noise of the user and generate sound data.

The step S120 of checking whether the surrounding environment of the user is suitable to evaluate the voice of the user includes the step of causing the server 10 to receive the generated sound data from the terminal 20.

The step S120 of checking whether the surrounding environment of the user is suitable to evaluate the voice of the user includes the step of causing the server 10 to receive the sound data from the terminal 20.

The step S120 of checking whether the surrounding environment of the user is suitable to evaluate the voice of the user includes a step of causing the server 10 to analyze the receive sound data to evaluate a level of noise around the user. For example, the server 10 may analyze the received sound data to measure a size of noise (for example, decibel, dB). That is to say, the method 100 in the present disclosure may determine that the recording environment is suitable when a size of noise around the user is less than a certain size.

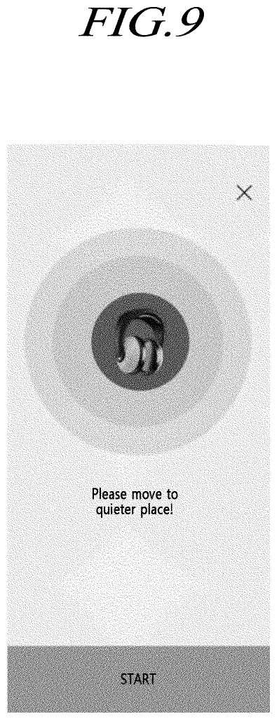

The step S120 of checking whether the surrounding environment of the user is suitable to evaluate the voice of the user includes a step of causing the server 10 to transmit information to the user terminal 20 regarding whether the surrounding environment is suitable to evaluate the voice of the user on the basis of the result of evaluating a level of noise. At this time, the method 100 in the present disclosure may cause the server 10 to cause the terminal 20 to guide the user to move to a place in which the surrounding environment is quiet so that the voice of the user may be recorded clearly and accurately. For example, as in FIG. 9, the method 100 in the present disclosure may cause the server 10 to cause the user terminal 20 to display the text “Please move to a quieter place!” on the display.

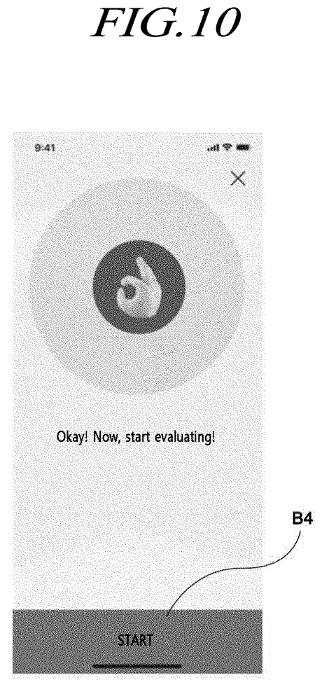

Also, as in FIG. 10, the method 100 in the present disclosure may cause the server 10 to display the text “Good! Now, start evaluating!” on the display of the user terminal 20 in response to the determining of the server 10 that the surrounding environment of the user is suitable for recording the voice of the user.

Moreover, as in FIG. 10, the method 100 in the present disclosure may further include a step of activating a start button B4 in response to the determining of the surrounding environment of the user to be suitable for recording the voice of the user.

The method 100 for evaluating dysarthria in the present disclosure may perform a step S130 of causing the server 10 to induce the user to perform at least one of sustained phonation, articulatory diadochokinesis, word reading, and sentence reading and record the voice of the user in response to the pressing of the user of the start button B4 after the step of activating the start button B4 of FIG. 10 to cause the user terminal 20 to perform sustained phonation evaluation. At this time, the server 10 may cause the user terminal 20 to display a fingerprint, instructions, and a recording button for the user to perform sustained phonation, articulatory diadochokinesis, word reading, and sentence reading. Here, the instructions may include content describing a specific task (action) to be performed by the user.

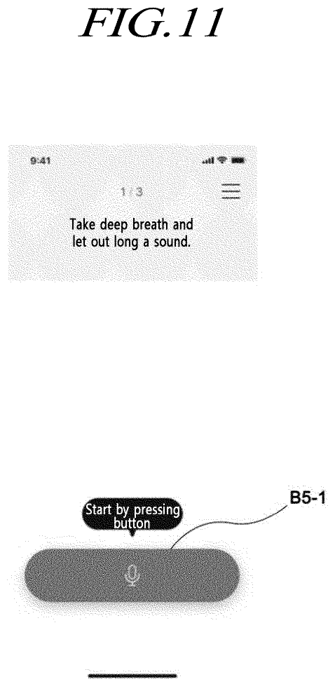

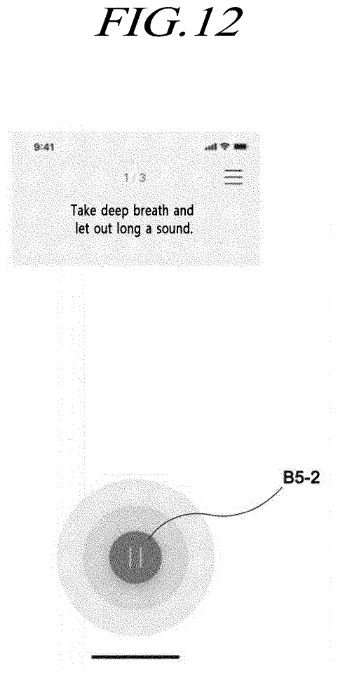

For example, as in FIG. 11, the server 10 may cause the user terminal 20 to display a recording button B5-1 on the display together with the instruction, “Take a deep breath and hold the ‘ah’ sound” so that the user can pronounce /ah/. In addition, as in FIG. 12, the method 100 in the present disclosure may cause the server 10 to cause the user terminal 20 to display a pause button B5-2 to pause recording in response to the pressing of the user of the record button B5-1 in FIG. 11.

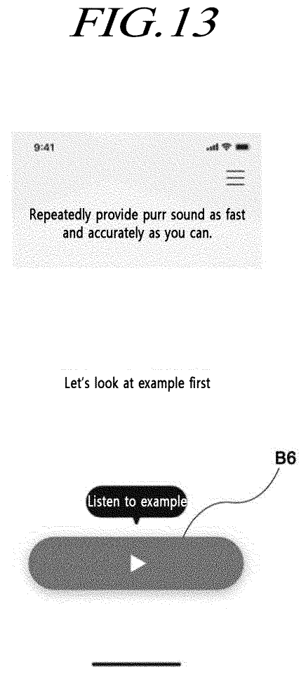

On the other hand, the method 100 in the present disclosure may further include a step of causing the server 10 to cause the user terminal 20 to output a sound to be pronounced by the user through a speaker. For example, as in FIG. 13, the method 100 in the present disclosure can cause the server 10 to cause the user terminal 20 to display an example listening button (B6) on the display together with the instruction “Listen to an example first.”

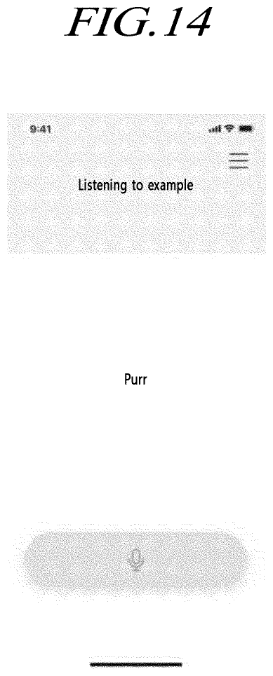

Also, as in FIG. 14, the method 100 in the present disclosure may cause the server 10 to cause the user terminal 20 to output the voice of “Purr” that the user needs to pronounce through the speaker.

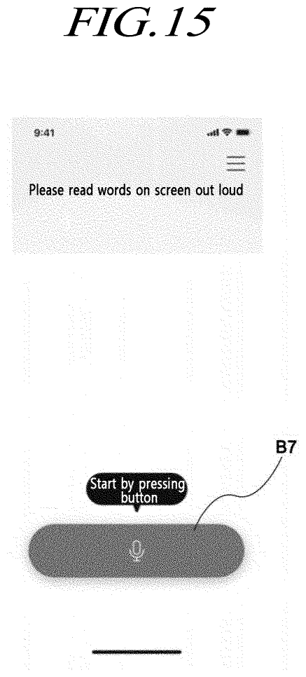

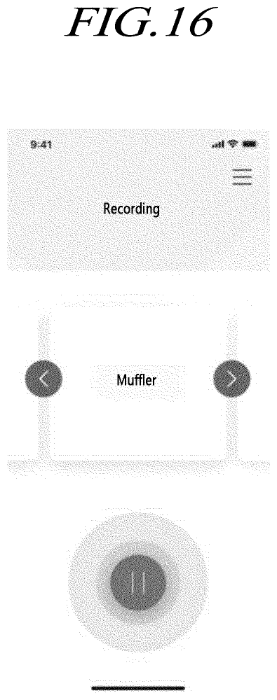

Moreover, as shown in FIG. 15 and FIG. 16, the method 100 for evaluating dysarthria in the present disclosure may cause the server 10 to induce the user to pronounce a word for sustained phonation evaluation by the user terminal 20 in response to the pressing of the user of the start button B4 after the step of activating the start button B4 in FIG. 10. For example, the method 100 in the present disclosure may cause the server 10 to cause the user terminal 20 to display the word “muffler” on the display as in FIG. 16 in response to the pressing of the user of a recording button B7 displayed on the display as in FIG. 15.

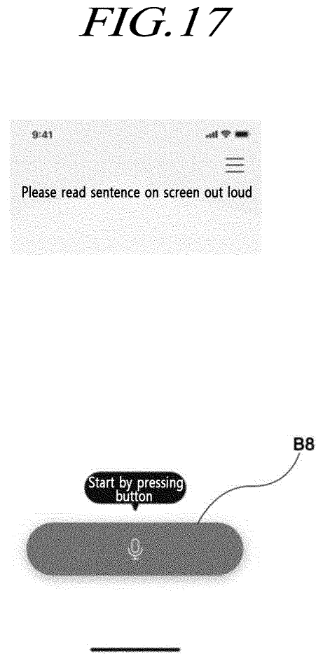

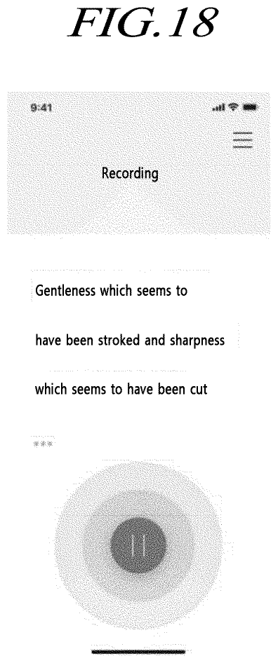

Furthermore, as shown in FIG. 17 and FIG. 18, the method 100 for evaluating dysarthria in the present disclosure may cause the server 10 to induce the user to pronounce a sentence for sustained phonation evaluation in response to the pressing of the user of the start button B4 in FIG. 10 after the step of activating the start button B4. For example, the method 100 in the present disclosure may cause the terminal 20 of the user to sequentially display on the display, as in FIG. 18, a message such as “Autumn in our country is truly beautiful. Above all, its outstanding beauty is felt even more when climbing a mountain. As you climb along the ridge which has a gentle slope which seems to be caressed and a sharp slope which seems to have been cut, you may not help but be amazed. The various colors such as red, blue, and yellow blend together to give a burning sensation and even make it mysterious. Lie down in the forest and look at the sky. If you look up at the white clouds which seem to be paired up and the high blue sky, you will surely understand why autumn has been called the season of paralysis of the heavens since ancient times.” At this time, the method 100 in the present disclosure may cause the server 10 to cause the user terminal 20 to display the words pronounced by the user among the entire sentence in a different color from the rest of the sentence when the user pronounces the sentence according to the fingerprint displayed on the display.

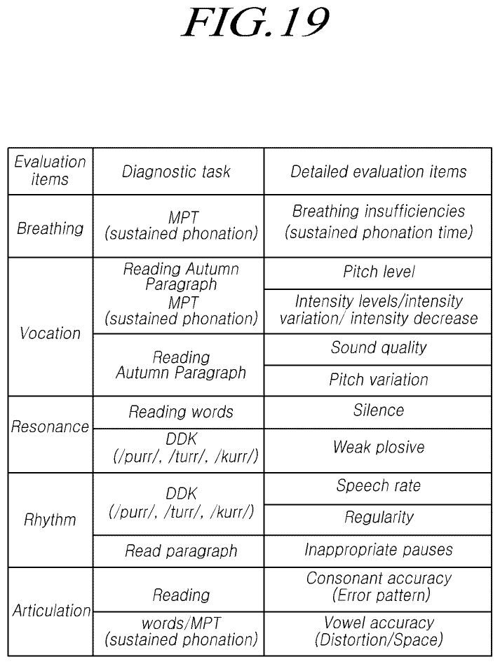

FIG. 19 is a table showing evaluation items, evaluation tasks, and detailed evaluation items used for performing the method 100 for evaluating dysarthria according to the example embodiment of the present disclosure.

Referring to the table in FIG. 19, the method 100 for evaluating dysarthria in the present disclosure includes a step S140 of causing the server 10 to evaluate a degree of dysarthria of the user on the basis of the recorded voice data. For example, as shown in the table in FIG. 19, the sustained phonation evaluation may include evaluating at least one of respiration of the user, phonation, resonance, prosody, and articulation. Here, the “autumn paragraph” in “Autumn Paragraph Reading” refers to a paragraph designed to be widely used for evaluating voice and speech such as sound quality, speech rate, and speech clarity and is an evaluation material in which various phonemes are evenly distributed (Autumn' paragraph (Kim Hyang-hee, 1996; <Appendix-1>)).

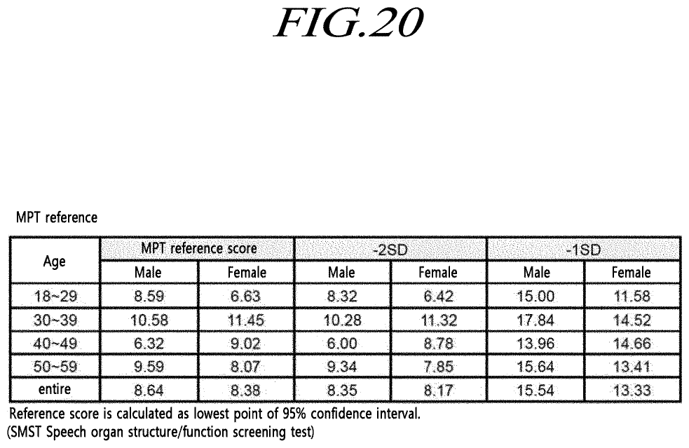

FIG. 20 is a table showing evaluation criteria for maximum phonation time (MPT) used for performing the method 100 for evaluating dysarthria according to an example embodiment of the present disclosure.

Referring again to FIG. 20 together with FIG. 12, the sustained phonation evaluation may include evaluating the degree of breathing insufficiency of the user, the level of breathing intensity, the degree of breathing intensity reduction, and the variation in breathing intensity on the basis of voice data recorded of the vocalization of the user to evaluate the breathing of the user. For example, as in FIG. 12, the method 100 for evaluating dysarthria may cause the server 10 to cause the terminal 20 to display the instruction, “Take a deep breath and make a long ‘ah’ sound,” while simultaneously recording the /ah/ pronunciation of the user.

For example, the method 100 for the present disclosure may cause the server 10 to cause the terminal 20 to display instructions on the display to guide the user to perform an action. The instructions may include “Press the record button at the bottom to start recording,” “Take a deep breath and pronounce the sounds /ah/, /ee/, and /oo/ for as long as you can at a comfortable pitch and loudness,” and “Repeat the pronouncement twice.” At this time, the method 100 for the present disclosure may cause the server 10 to cause the terminal 20 to measure the vocalization time (unit: seconds) while the user repeatedly performs vocalization. Then, the maximum value of the maximum phonation time is calculated.

For example, as shown in the table of FIG. 20, in order to evaluate the respiratory function of the user, the method 100 in the present disclosure may evaluate the respiratory function of the user on the basis of the reference score of maximum phonation time (maximum phonation time, MPT).

On the other hand, the sustained phonation evaluation may include evaluating pitch level, vocalization intensity level, sound quality, and pitch variations to evaluate the vocalization of the user.

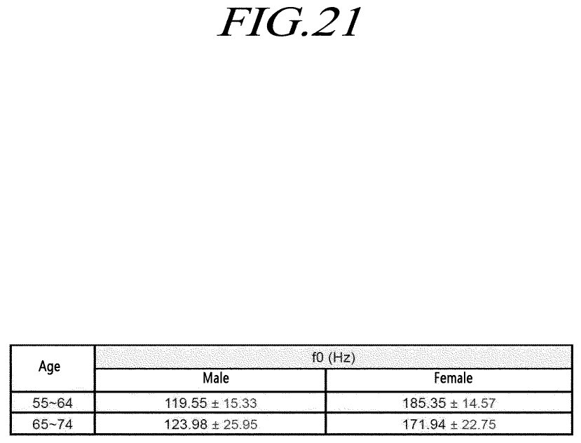

FIG. 21 is a table showing evaluation criteria for a pitch level of vocalization used for performing the method 100 for evaluating dysarthria according to an example embodiment of the present disclosure.

For example, as shown in the table of FIG. 21, the method 100 in the present disclosure may cause the server 10 to measure the voice frequency (Hz) of a 2 to 3 second section excluding the first 1 second of the voice for the first /ah/ spoken by the user to evaluate the pitch level of the voice of the user. The method 100 in the present disclosure may cause the server 10 to determine whether the measured voice frequency Hz is normal on the basis of the criteria according to an age and a gender shown in the table of FIG. 21. Furthermore, the method 100 in the present disclosure may cause the server 10 to analyze a voice tilt value when measuring a change in pitch of the voice of the user.

For example, the method 100 in the present disclosure may cause the server 10 to perform distance setting and voice calibration between the mouth of the user and the microphone before evaluating the intensity level of the voice of the user. For example, the method 100 in the present disclosure may cause the server 10 to evaluate how much the pitch of the voice decreases or the sound becomes quieter while the user is specking /ah/ to evaluate the level of intensity of the vocalization of the user.

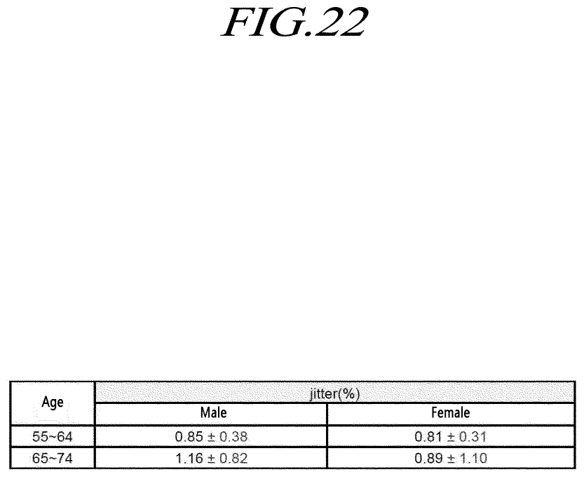

FIG. 22 is a table showing evaluation criteria for pitch variations used for performing the method 100 for evaluating dysarthria according to an example embodiment of the present disclosure.

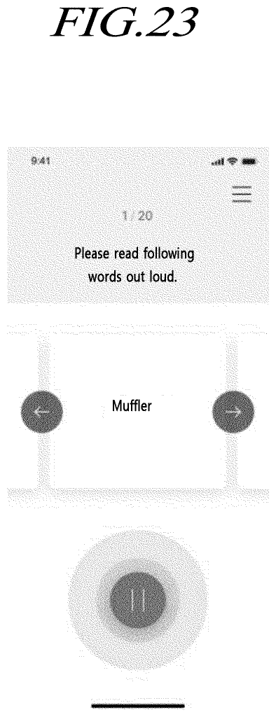

For example, the method 100 in the present disclosure may cause the server 10 to extract jitter and Shimmer values using a voice analysis program (for example, Praat tool) on the basis of recorded data of the speech of the user to evaluate the pitch variations of the user. The method 100 in the present disclosure may cause the server 10 to determine whether the extracted jitter and Shimmer values of the user are normal on the basis of the jitter (%) criteria of the table of FIG. 23.

FIG. 23 is an image showing the details of the display of the terminal 20 in which the method 100 for evaluating dysarthria according to an example embodiment of the present disclosure is used.

Referring to FIG. 23, the sustained phonation evaluation of the method 100 in the present disclosure may include evaluating the degree of nasality and the degree of weak plosives to evaluate the resonance of the user.

The method 100 in the present disclosure may instruct the server 10 to pronounce a word in order to evaluate the level of rudeness of the user. For example, as shown in FIG. 23, the method 100 in the present disclosure may cause the server 10 to cause the terminal 20 to display, on the display of the terminal 20, a plurality of words such as “muffler.” along with an instruction “Please read the following words aloud.”

The method 100 in the present disclosure may cause the server 10 to determine whether nasalization (voiced sounds are produced as nasal sounds) occurring among the vowel errors in the words pronounced by the user to evaluate the degree of nasality of the user. For example, the method 100 in the present disclosure may cause the server 10 to determine that the /Horanyi/ pronounced by the user corresponds to the recording of /Honangyi/ with a strong nasal sound. Here, ‘nasalization’ refers to cases in which non-nasal phonemes are pronounced by replacing them with nasal sounds (mi-eum, ni-eun, and lee-eung).

The method 100 in the present disclosure may cause the server 10 to evaluate the voice quality of the speech of the user by evaluating the voice quality of the table in the drawing on a scale of 0 to 3 on the basis of voice data recorded through the speaking of the user of a paragraph. Here, 0 represents normal, 1 represents mild, 2 represents moderate, and 3 represents severe. Specifically, the method 100 in the present disclosure may cause the server 10 to evaluate the fundamental frequency, pitch variations, intensity variations, and noise level of the voice of the user to evaluate the voice quality of the user.

The method 100 in the present disclosure may cause the server 10 to instruct the terminal 20 to perform a single syllable repetition task (Alternating Movement Rate, AMR) or movement speed (Sequential motion rate, SMR) to evaluate the degree of weak plosive sounds of the user. For example, the method 100 in the present disclosure may cause the server 10 to verify whether /pi-eup/, /ti-eut/, and /ki-euk/ are pronounced correctly as plosives by transcribing the consonants of a single or long syllable pronounced by the user. For example, the method 100 in the present disclosure may cause the server 10 to determine that the sound strength is weakened when the user's single or long syllables /pi-eup/, /ti-eut/, and /ki-euk/ are pronounced as /bi-eup/, /di-geut/, and /gi-yeok/.

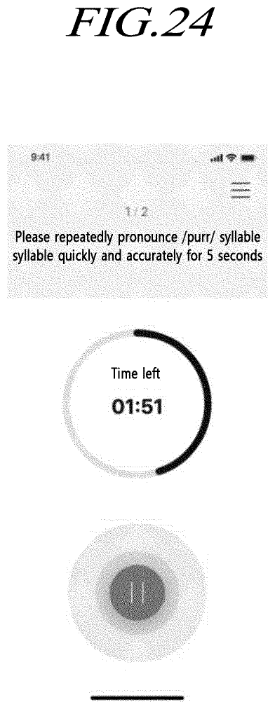

FIG. 24 is images showing the details of the display of the terminal 20 in which the method 100 for evaluating dysarthria according to an example embodiment of the present disclosure is used.

Referring to FIG. 24, the sustained phonation evaluation may include evaluating speech rate, regularity, and inappropriate pauses to evaluate the rhyme of the user. As in FIG. 24, the method 100 in the present disclosure may cause the server 10 to record the voice of the user along with the instruction “Please repeatedly pronounce the /purr/ syllable quickly and accurately for 5 seconds” to the terminal 20. At this time, when evaluating the AMR task, the user can pronounce one syllable of /purr/, /turr/, or /kurr/, or three syllables of /purr-turr-kurr/, three times as fast as possible. Alternatively, when evaluating sound rate of movement (SMR), the user can pronounce one syllable of /purr/, /turr/, or /kurr/, or three syllables of /purr-turr-kurr/, three times as fast as possible. The method 100 in the present disclosure may calculate the number of repetitions per second by dividing the number of repetitions of 1 syllable or 3 syllables by the server 10 by 5 seconds. The method 100 in the present disclosure causes the server 10 to calculate an average of the three times after performing the method three times.

The method 100 in the present disclosure may enable the server 10 to provide the terminal 20 with a sentence and instructions to be pronounced by the user and record the voice of the user to evaluate the speech speed of the paragraph. Based on the voice data recorded and generated in this way, the method 100 in the present disclosure may cause the server 100 to evaluate the speech speed of the paragraph of the user in accordance with a predetermined standard. For example, the method 100 in the present disclosure may cause the server 10 to measure syllables per second (SPS) and syllables per minute (SPM) on the basis of voice data recorded in paragraphs. At this time, the method 100 in the present disclosure may cause the server 10 to evaluate that the ability regarding speech speed is low in response to the syllables per second (SPS) and syllables per minute (SPM) of the user falling below the normal standard.

The method 100 in the present disclosure may be used for causing the server 10 to evaluate the regularity of the rhyme of the user by analyzing how regularly and rhythmically each syllable of /purr/, /turr/, /kurr/, and /purr-turr-kurr/ is spoken when repeatedly performed. For example, the method 100 in the present disclosure may cause the server 10 to evaluate the degree to which the period between syllables is constant by dividing the voice spoken by the user into syllables (segmentation). For example, the method 100 in the present disclosure may cause the server 10 to determine whether there is a drift between syllables by checking the distribution of duration between syllables.

The method 100 in the present disclosure may cause the server 10 to analyze whether an inappropriate pause is present and a frequency thereof by comparing the interval of the pause of the user with the interval of a correct pause (break index) on the basis of voice data generated through the speaking of the user of a syllable to evaluate inappropriate pauses in the rhyme of the user. For example, the method 100 in the present disclosure may cause the server 10 to analyze inappropriate sections and frequencies by indicating correct accented phrases (Accentual Phrase (AP)) and intonation phrases (Intonation Phrase (IP)) in a paragraph spoken by the user.

On the other hand, the sustained phonation evaluation of the method 100 in the present disclosure may include evaluating consonant accuracy and vowel accuracy to evaluate the articulation of the user. The method 100 in the present disclosure may cause the server 10 to perform analysis of the consonant accuracy (Total PCC) and phonological error pattern of the target word to evaluate the consonant accuracy of the user. For example, the method 100 in the present disclosure may include a phoneme recognition module which causes the server 10 to check how the user pronounced /Horanyi/ by dividing it into “hi-eut,” “oh,” “ri-eul,” “ah,” “'ng,” “ng,” and “ee” to evaluate the consonant accuracy of the user.

For example, the method 100 in the present disclosure may cause the server 10 to obtain the overall consonant accuracy by calculating the ratio of the consonants which are correctly pronounced among all consonants included in words pronounced by the user as a percentage as in Expression 1 which will be described below. At this time, the method 100 in the present disclosure may cause the server 10 to determine whether the target word in the table pronounced by the user is an error consonant on the basis of whether the pronunciation of the word in the table matches the transcription pronunciation.

$$Total consonant accuracy = ( 94 - number of error consonants ) / 94 \times 100 \qquad Expression 1$$

The method 100 in the present disclosure may cause the server 10 to evaluate the distorted vowels of the user to evaluate the pronunciation accuracy of the user. The method 100 in the present disclosure may cause the server 10 to evaluate the distorted vowels of the user to evaluate the pronunciation accuracy of the user. For example, the method 100 in the present disclosure may cause the server 10 to analyze the vowel space when the user speaks to evaluate vowel distortion and perform an analysis on why some parts of the space in which the vowel is pronounced are not possible. That is to say, the method 100 in the present disclosure may cause the server 10 to evaluate vowel distortion, speech intelligibility, or the like by analyzing the size of the vowel space compared to a normal user.

Thus, the method 100 for evaluating dysarthria in the present disclosure may effectively evaluate a degree of dysarthria of the user by including a step S140 of evaluating a degree of dysarthria of the user on the basis of recorded voice data.

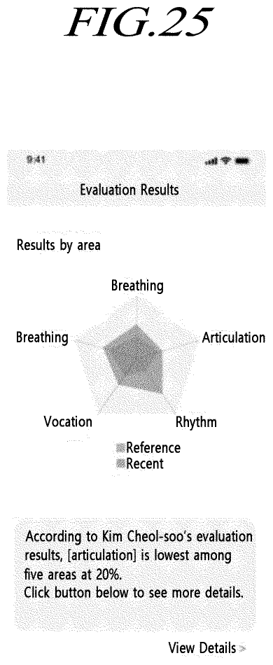

FIGS. 25 to 40 are images showing the details of an evaluation result report 30 provided in the method 100 for evaluating dysarthria according to an example embodiment of the present disclosure.

Referring to FIGS. 25 to 40, the method 100 for evaluating dysarthria according to an example embodiment of the present disclosure further includes, after the step S140 of evaluating the degree of dysarthria of the user on the basis of the recorded voice data, a step of causing the server 10 to transmit the evaluation result report 30 including evaluation result information evaluating a degree of dysarthria of the user to the user terminal 20 and a step of causing the server 10 to cause the user terminal 20 to display the evaluation result report 30 on the display and report it to the user.

For example, as in FIG. 25, the evaluation result report 30 may graphically display the levels for each area of the respiration of the user, phonation, resonance, prosody, and articulation. For example, as in FIG. 25, the evaluation result report 30 may be displayed in a graph by comparing the existing (past) results and recent results for each of the respiration of the user, phonation, resonance, prosody, and articulation areas.

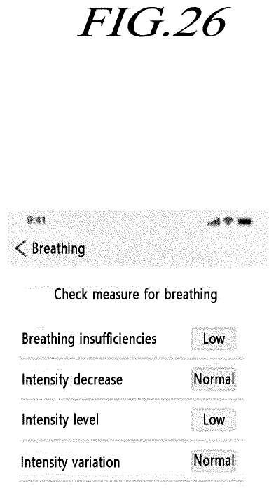

The evaluation result report 30 may include content classifying the evaluation results of at least one of the respiration of the user, phonation, resonance, prosody, and articulation as high, medium, or low. For example, as shown in FIG. 26, the evaluation result report 30 may indicate the evaluation results of each of the breathing insufficiencies of the breathing of the user, intensity decreases, intensity levels, and intensity variations as high, normal, or low.

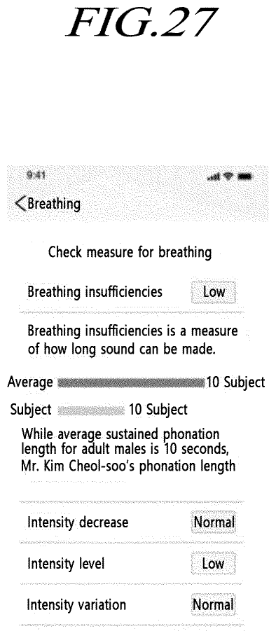

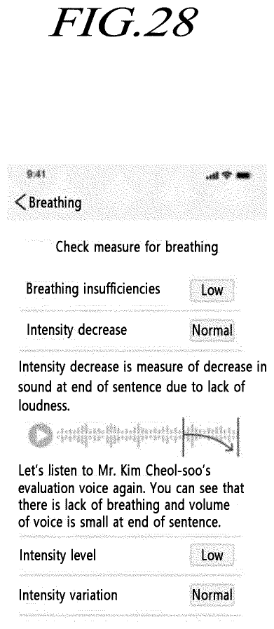

The evaluation result report 30 may include a graph showing the evaluation results for at least one of the respiration of the user, phonation, resonance, prosody, and articulation. For example, as in FIG. 27, the evaluation result report 30 may include a result comparing the average value and the level of the user for the evaluation of the breathing insufficiencies of the breathing of the user. For example, as in FIG. 28, the evaluation result report 30 may include a graph showing the meaning of evaluation items of a decrease in the intensity of the breathing of the user and the portion of the voice data where the intensity is decreased.

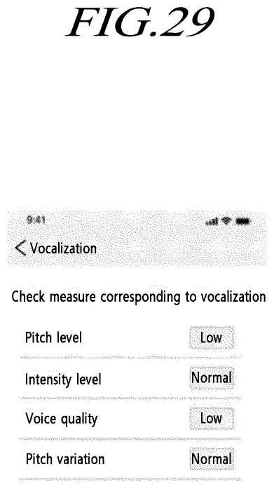

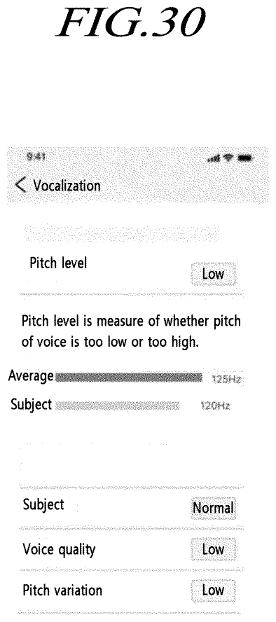

For example, as in FIG. 29, the evaluation result report 30 may express the evaluation results of each of the pitch level, intensity level, sound quality, and pitch variations of the voice of the user as high, medium, or low. For example, as shown in FIG. 30, the evaluation result report 30 may include a graph comparing the meaning of evaluation items of the pitch level of the user's pronunciation and the average value with the pitch level of the user.

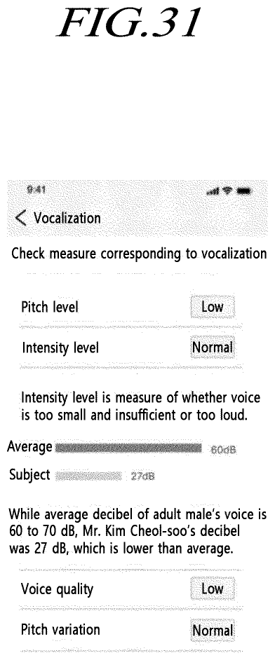

For example, as shown in FIG. 31, the evaluation result report 30 may include a graph comparing the meaning of evaluation items of the intensity level of the voice of the user and the average value with the intensity level of the user.

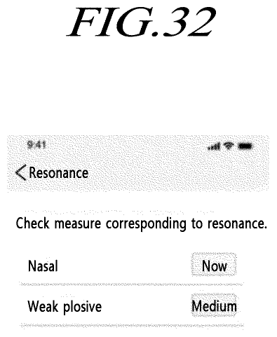

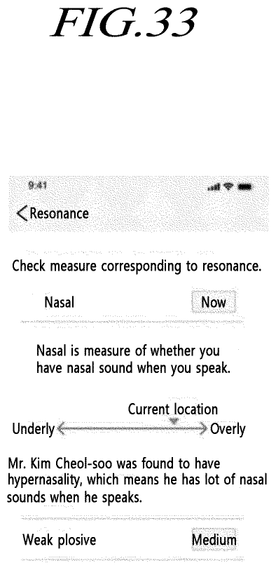

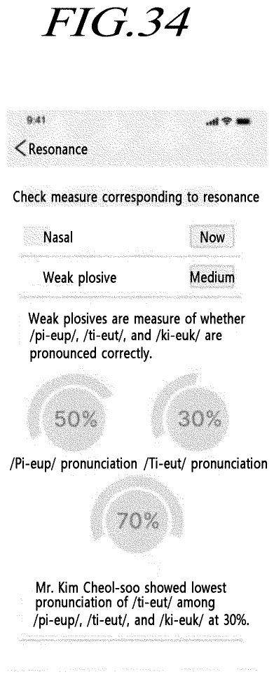

For example, as in FIG. 32, the evaluation result report 30 may express the evaluation results of the resonance of the user, nasal sound and weak plosive sound as high, medium, or low, respectively. For example, as in FIG. 33, the evaluation result report 30 may include a graph showing the meaning of evaluation items of the resonance of the user and the current resonance level of the user among under and over. For example, as in FIG. 34, the evaluation result report 30 may include a circle graph showing the meaning of evaluation items of the resonance weak plosives of the user and the current weak plosive level of the user (%).

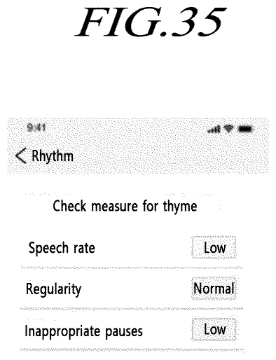

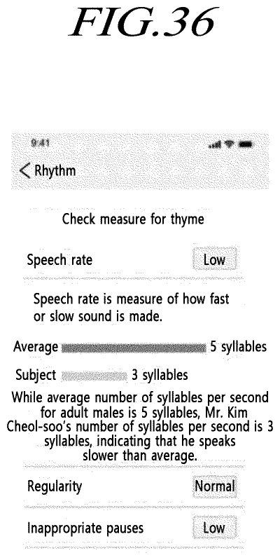

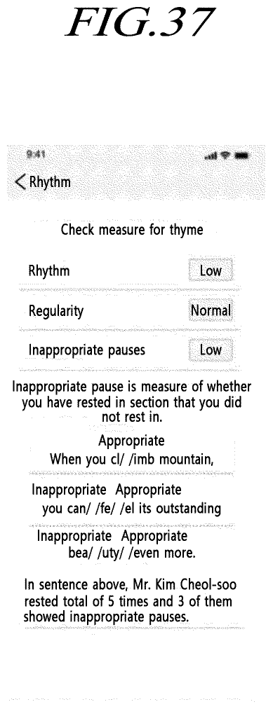

For example, as in FIG. 35, the evaluation result report 30 may indicate the evaluation results of each of the speech rate of the user, regularity, and inappropriate pauses as high, average, or low. For example, as in FIG. 36, the evaluation result report 30 may include a graph comparing the meaning of evaluation items of the rhyme speech rate of the user and the average value with the speech rate level of the user. For example, as shown in FIG. 37, the evaluation result report 30 may include the meaning of evaluation items of inappropriate pauses in the rhyme of the user and text indicating the part of the sentence pronounced by the user in which the inappropriate pause occurs.

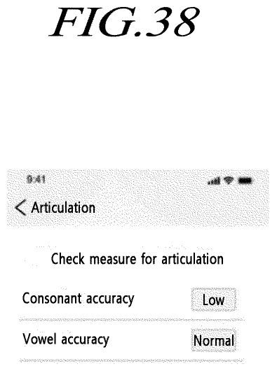

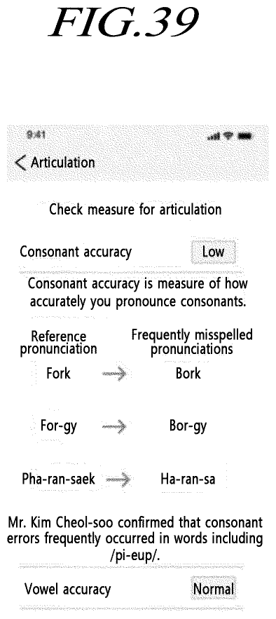

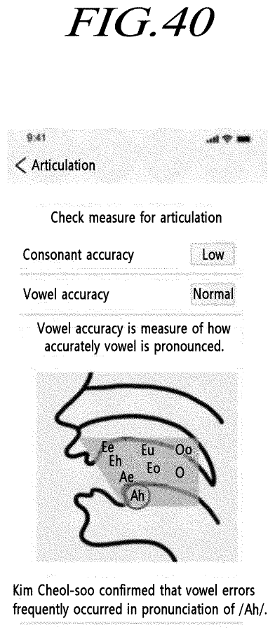

For example, as in FIG. 38, the evaluation result report 30 may express the evaluation results of each of the consonant accuracy and vowel accuracy of the pronunciation of the user as high, normal, or low. For example, as in FIG. 39, the evaluation result report 30 may include text indicating the meaning of evaluation items of the consonant accuracy of the pronunciation of the user and frequently misspelled consonants. For example, as in FIG. 40, the evaluation result report 30 may include the meaning of evaluation items of the vowel accuracy of the articulation of the user, text indicating frequently misspelled vowels, and an intraoral picture indicating the location of incorrect vowels.

Also, the server 10 may evaluate the degree of dysarthria of the user and provide the results to the medical staff. The medical staff may provide the diagnosis or opinion of the medical staff to the server 10 on the basis of the results of evaluating a degree of dysarthria. The server 10 may generate feedback to be provided to the user on the basis of the diagnosis or opinion of the medical staff.

The method 100 for evaluating dysarthria according to another example embodiment of the present disclosure may further include a step of causing the server 10 to cause the user terminal 20 to conduct a questionnaire to evaluate the psychological state of the user before the step S120 of checking whether the surrounding environment of the user is appropriate for evaluating the voice of the user.

Furthermore, the method 100 for evaluating dysarthria according to another example embodiment of the present disclosure may include a step of causing the server 10 to cause the terminal 20 to provide a questionnaire to the user, a step of causing the server 10 to cause the terminal 20 to receive a response from the user, and a step of causing the server 10 to cause the terminal 20 to store a response received from the user in the memory or transmit the response to the server 10. The questionnaire may be a questionnaire received from the server 10 and stored in the terminal 20.

Referring again to FIG. 1, a dysarthria evaluation device according to an example embodiment of the present disclosure may be a computing device including a processor and a memory. For example, the dysarthria evaluation device may be the server 10 which is capable of communicating with the terminal 20.

For convenience of description herein, although the present disclosure describes various functions for evaluating dysarthria as being performed on the terminal 20 using the processor of the server 10; this is not limited to the present disclosure and some functions for evaluating dysarthria may be performed using the processor of the terminal 20 itself. For example, the processor of the terminal 20 may directly perform a step of evaluating a degree of dysarthria of the user on the basis of voice data generated by recording the voice of the user received through a microphone.

For example, the processor of the terminal 20 may control objects to be displayed to change in response to the voice of the user being received through the microphone.

The device and the method 100 described above may be implemented as hardware components, software components, and/or a combination of hardware components and software components. For example, the device and the constituent elements described in the embodiments may be implemented using one or more general-purpose computers or special-purpose computers such as, for example, a processor, a controller, an arithmetic logic unit (ALU), a digital signal processor, a microphone, a field programmable array (FPA), a programmable logic unit (PLU), a microphone processor, or any other device capable of executing instructions and responding. A processing device may execute an operating system (OS) and one or more software applications running on the operating system. Furthermore, the processing device may also access, store, manipulate, process and generate data in response to the execution of the software. For ease of understanding, the processing device is sometimes described as being used alone, but those skilled in the art will recognize that the processing device may include a plurality of processing elements and/or a plurality of types of processing elements. For example, a processing device may include a plurality of processors or a processor and a controller. Moreover, other processing configurations such as parallel processors are also possible.

Software may include computer programs, codes, instructions, or a combination of one or more of these and may configure processing devices to do what is desired or may command processing devices, either independently or collectively. The software and/or data may be permanently or temporarily embodied in any type of machine, component, physical device, virtual apparatus, computer storage media or device, or transmitted signal waves, for interpretation by a processing device or for providing instructions or data to a processing device. The software may be distributed across networked computer systems and stored or executed in a distributed method 100. The software and the data may be stored on one or more computer-readable recording media.

The described embodiments of the present disclosure may also be practiced in distributed computing environments in which certain tasks are performed through remote processing devices which are linked to communicate with over a network. In a distributed computing environment, program modules may be located in both local and remote memory storage devices.

Although the embodiments have been described with limited drawings as described above, those skilled in the art can apply various technical modifications and variations on the basis of the above description. For example, suitable results may be achieved even if the described techniques are performed in a different order than in the described method 100 and/or components of the described system, structure, device, circuit, or the like are combined or combined in a different manner than in the described method 100 or are replaced or substituted by other components or equivalents.

Therefore, other implementations, other embodiments, and equivalents to the claims are also included in the scope of the claims which will be described below.

### REFERENCE SIGNS LIST

10: Server
20: Terminal
100: Method for evaluating dysarthria
S110 to S140: Steps of method for evaluating dysarthria
30: Evaluation result report

1-13. (canceled)

14. A method for evaluating dysarthria by which a server receives voice data of a user from a user terminal and evaluates dysarthria of the user, wherein
the server includes a processor and a database and communicates with the terminal, and
the terminal includes a processor, a memory, a display, and a microphone and communicates with the server,
the method comprises wherein the server:
causing the terminal to describe a dysarthria evaluation process to the user;
causing the user terminal to check whether the surrounding environment of the user is suitable to evaluate the voice of the user;
causing the user terminal to induce to perform at least one from among sustained phonation, articulatory diadochokinesis, word reading, and sentence reading for sustained phonation evaluation, and record the voice of the user; and
evaluating a degree of dysarthria of the user on the basis of the recorded voice data, and the sustained phonation evaluation includes
evaluating a degree of breathing insufficiency of the user, a level of breathing intensity, a degree of breathing intensity reduction, and a variation in breathing intensity to evaluate the breathing of the user.

15. The method for evaluating dysarthria according to claim 14, wherein the checking whether the surrounding environment of the user is suitable to evaluate the voice of the user includes
causing the server to cause the terminal to record noise around the user and generate sound data;
causing the server to receive the generated sound data from the terminal;
causing the server to receive the sound data from the terminal;
causing the server to analyze the received sound data to evaluate a level of noise around the user; and
causing the server to transmit, to the user terminal, information regarding whether the surrounding environment is suitable to evaluate the voice of the user on the basis of the result of evaluating the level of noise.

16. The method for evaluating dysarthria according to claim 14, wherein the sustained phonation evaluation includes
evaluating a pitch level, a vocalization intensity level, sound quality, and pitch variations to evaluate the vocalization of the user.

17. The method for evaluating dysarthria according to claim 14, wherein the sustained phonation evaluation includes
evaluating a degree of nasality and weak plosives to evaluate the resonance of the user.

18. The method for evaluating dysarthria according to claim 14, wherein the sustained phonation evaluation includes
evaluating a speech rate, regularity, and inappropriate pauses to evaluate the rhyme of the user.

19. The method for evaluating dysarthria according to claim 14, wherein the sustained phonation evaluation includes
evaluating consonant accuracy and vowel accuracy to evaluate the articulation of the user.

20. A method for evaluating dysarthria by which a server receives voice data of a user from a user terminal and evaluates dysarthria of the user, wherein
the server includes a processor and a database and communicates with the terminal, and
the terminal includes a processor, a memory, a display, and a microphone and communicates with the server,
the method comprises in which the server: causing the terminal to describe a dysarthria evaluation process to the user;
causing the user terminal to check whether the surrounding environment of the user is suitable to evaluate the voice of the user;
causing the user terminal to induce to perform at least one from among sustained phonation, articulatory diadochokinesis, word reading, and sentence reading for sustained phonation evaluation, and record the voice of the user; and
evaluating a degree of dysarthria of the user on the basis of the recorded voice data, and before checking whether the surrounding environment of the user is suitable to evaluate the voice of the user,
further includes causing the server to cause the user terminal to conduct a questionnaire to evaluate a psychological state of the user.

21. The method for evaluating dysarthria according to claim 14, further comprising:
after the step of evaluating a degree of dysarthria of the user on the basis of the recorded voice data,
causing the server to transmit an evaluation result report including evaluation result information obtained by evaluating a degree of dysarthria of the user to the user terminal; and
causing the server to cause the user terminal to display the evaluation result report on the display and report the evaluation result report to the user.

22. The method for evaluating dysarthria according to claim 21, wherein the evaluation result report includes
the contents of classifying the ability of at least one of the respiration of the user, phonation, resonance, prosody, and articulation as high, average, or low.

23. The method for evaluating dysarthria according to claim 21, wherein the evaluation result report includes
a graph showing the ability of at least one of the respiration of the user, phonation, resonance, prosody, and articulation.

24. The method for evaluating dysarthria according to claim 21, wherein the evaluation result report includes
the contents indicating a port of the sentence read by the user in which inappropriate pauses appear to evaluate the inappropriate pauses in the rhyme of the user.

25. A dysarthria evaluation device performing a method for evaluating dysarthria according to claim 14, comprising:
a processor; and
a memory.

26. The method for evaluating dysarthria according to claim 20, wherein the sustained phonation evaluation includes
evaluating at least one of the respiration of the user, phonation, resonance, prosody, and articulation.
